(19) United States
(12) Patent Application Publication
(10) Pub. No.: US 2007/0299139 A1
(43) Pub. Date: Dec. 27, 2007

(54) Lyophilisate Comprising N-Diaminomethylene-2-Methyl-4,5-Di(Methylsulfonyl)Benzamide

(76) Inventors: Hanns-Christian Mahler, Wiesbaden (DE); Rita Heckmann, Princeton, NJ (US); Dorothee Kress, Darmstadt (DE); Dirk Schiroky, Pfungstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/575,741

(22) PCT Filed: Sep. 10, 2005
(86) PCT No.: PCT/EP05/09741

§ 371 Date: Mar. 21, 2007

(30) Foreign Application Priority Data
Sep. 22, 2004 (DE) 10 2004 045 825.1

(51) Int. Cl. A61K 31/166; A61P 9/10; C07C 211/44

(52) U.S. Cl. 514/646; 564/440

(57) ABSTRACT

The invention relates to a lyophilisate comprising N-diaminomethylene-2-methyl-4,5di(methylsulfonyl)benzamide as active compound. The composition has increased storage stability, even at elevated temperatures, and can be administered parenterally as medicament after reconstitution.

The present invention relates to a stable lyophilisate comprising N-diaminomethylene-2-methyl-4,5-di(methylsulfonyl)benzamide as active compound and to the preparation of the lyophilisate.

N-diaminomethylene-2-methyl-4,5-di(methylsulfonyl)benzamide is a highly effective and selective inhibitor of the sodium/proton exchanger (NHE-1) having cardioprotective actions. The active compound is intended to be employed firstly for pre-, peri- and postoperative myocardial protection during operations on high-risk patients.

The development of this active compound in intensive medicine requires that it is available in parenterally administrable form, best as aqueous solution. However, extensive experiments have shown that N-diaminomethylene-2-methyl-4,5-di(methylsulfonyl)benzamide is not stable in aqueous solution, but instead hydrolyses.

One way of stabilising active compounds is freeze drying of solutions comprising the active compound. It should be possible to reconstitute the lyophilisates obtained by freeze drying by addition of an aqueous solvent to a parenterally administrable active-compound solution, enabling the latter to be made available at short notice and in a simple manner if required. Extensive experiments with the active compound alone and with various adjuvants/bulking agents, such as, for example, mannitol, glucose, sodium acetate, glycine, dextran, lactose, sucrose, calcium gluconate and urea, gave physically and chemically unstable products if the parameters according to the invention were not taken into account. Unstable product cakes which were difficult to reconstitute and in addition often resulted in solutions containing particles on reconstitution were regularly obtained. The solutions obtained from the lyophilisates likewise comprised decomposition products of diaminomethylene-2-methyl-4,5-di(methylsulfonyl)benzamide, meaning that overall it was not possible to achieve stabilisation of the active compound.

The object of the present invention was to provide a stabilised composition for diaminomethylene-2-methyl-4,5-di(methylsulfonyl)benzamide. The composition should comprise no toxicologically unacceptable adjuvants, should be stable for an extended time under increased stress conditions, such as elevated temperature and atmospheric humidity, and should easily be reconstitutable with an aqueous solvent to give a parenterally administrable solution.

Surprisingly, it was possible to provide a composition which meets these requirements by freeze drying an aqueous, optionally buffered solution which comprises diaminomethylene-2-methyl-4,5-di(methylsulfonyl)benzamide and a bulking agent and has a pH of 1 to 3.8. The present invention therefore relates to a lyophilisate obtainable by freeze drying an aqueous solution which comprises at least N-diaminomethylene-2-methyl-4,5-di-(methylsulfonyl)benzamide and a bulking agent and has a pH of 1 to 3.8. It is preferred for the aqueous solution used for the freeze drying to have a pH of 2.5 to 3.5, particularly preferably a pH of about 3.

The composition according to the invention is physiologically well tolerated, can be prepared easily, can be dispensed precisely and is stable with respect to assay, decomposition products and aggregates over the duration of storage and also after repeated freezing and thawing operations. It can be stored in a stable manner over a period of 2 years at refrigerator temperature (2-8° C.) and at room temperature (23-27° C., 60% relative atmospheric humidity (RH). Surprisingly, the composition according to the invention can also be stored in a stable manner over the said period at elevated temperatures and atmospheric humidities, for example at a temperature of 40° C. and 75% RH.

The lyophilisate can be reconstituted in a simple manner to give a ready-to-administer particle-free solution by addition of an aqueous solvent, for example water for injection purposes or an isotonic aqueous solution. The reconstituted solution is stable over a period of about 7 days, but is particularly preferably administered within 24 hours.

Diaminomethylene-2-methyl-4,5-di(methylsulfonyl)benzamide-containing solutions having a pH of 1 to 3.8, preferably having a pH of 2.5 to 3.5, particularly preferably having a pH of 3, and an osmolality of 50-500 mOsmol/kg can advantageously be prepared from the composition according to the invention by reconstitution with aqueous solvents. The reconstituted composition can then be administered directly intravenously, intraarterially and also subcutaneously substantially without pain. In addition, the composition can also be added to infusion solutions, such as, for example, glucose solution, isotonic saline solution or Ringer's solution, which may also comprise further active compounds, also enabling larger amounts of active compound to be administered. The lyophilisate is preferably taken up in approximately 5% glucose solution, 0.9% sodium chloride solution or Ringer's lactate solution.

The pH of the aqueous solution used for freeze drying is advantageously adjusted using a physiologically tolerated organic or inorganic acid. Acids which are suitable for this purpose are, for example, citric acid, phosphoric acid, sulfuric acid, acetic acid, formic acid and hydrochloric acid. The pH is preferably adjusted using hydrochloric acid.

The lyophilisate according to the invention may comprise, as bulking agents, sugar alcohols, sugars, urea, glutamic acid and/or the substance class of the ectoines and hydroxyectoines, i.e. preferably substances which are obtained as amorphous substances having a glass transition temperature above 20° C. on freeze drying. The lyophilisate preferably comprises sugars and/or sugar alcohol(s) as bulking agents. Sugars which can be employed are mono-, di- or trisaccharides. These sugars can be employed either alone or mixed with sugar alcohols. Monosaccharides which may be mentioned by way of example are glucose, mannose, galactose, fructose and sorbose, disaccharides which may be mentioned by way of example are sucrose, lactose, maltose or trehalose, and a trisaccharide which may be mentioned by way of example is raffinose. Sugar alcohols which can be employed in accordance with the invention are, for example, mannitol and sorbitol. Sucrose, lactose, maltose, trehalose mannitol and/or sorbitol are preferably present, mannitol and/or lactose are particularly preferred. These bulking agents are generally present in the aqueous solution to be freeze dried in a concentration of 0.01 to 0.20 mmol/l, preferably in a concentration of 0.03 to 0.12 mmol/l.

After reconstitution with the proposed volume of solvent, the bulking agent is present in the resultant solution in a concentration of 0.005 to 0.23 mmol/l. The bulking agent is preferably present in the reconstituted solution in a concentration of 0.03 to 0.12 mmol/l.

The lyophilisate according to the invention may comprise one or more buffers. Buffers which can be employed are basically all physiologically tolerated substances which are suitable for setting the desired pH. The amount of buffer substance here is selected so that the aqueous solution obtained after reconstitution of the lyophilisate, for example with water for injection purposes, has a buffer concentration of 5 mmol/l to 50 mmol/l, preferably 10 to 20 mmol/l. Preferred buffers are citrate buffers and/or phosphate buffers. Suitable phosphate buffers are solutions of the mono- and/or disodium and potassium salts of phosphoric acid, such as disodium hydrogenphosphate or potassium dihydrogenphosphate, and mixtures of the sodium and potassium salts, such as, for example, mixtures of disodium hydrogenphosphate and potassium dihydrogenphosphate.

In addition, the lyophilisates according to the invention may comprise further physiologically tolerated adjuvants, such as, for example, antioxidants, such as ascorbic acid or glutathione, preservatives, such as phenol, m-cresol, methyl- or propylparaben, chlorobutanol, thiomersal or benzalkonium chloride, stabilisers, bulking agents, such as, for example, sucrose, lactose, maltose, trehalose, mannitol and/or sorbitol, mannitol and/or lactose and solubilisers, such as polyethylene glycols (PEG), for example PEG 3000, 3350, 4000 or 6000, or cyclodextrins, for example hydroxypropyl-β-cyclodextrin, sulfobutylethyl-β-cyclodextrin or γ-cyclodextrin, or dextrans. In accordance with an advantageous embodiment, the lyophilisate according to the invention comprises one or more physiologically tolerated adjuvants selected from the group consisting of antioxidants, preservatives and/or solubilisers.

The composition according to the invention can be prepared by preparing an aqueous composition which comprises at least N-diaminomethylene-2-methyl-4,5-di(methylsulfonyl)benzamide and a bulking agent, if necessary adjusting the resultant solution to a pH of 1 to 3.8 using an acid and subsequently lyophilising it. The aqueous solution comprising active compound(s) and adjuvant(s) is advantageously also sterile-filtered before freeze drying.

The lyophilisate obtained can be reconstituted by addition of an aqueous solvent to give an aqueous composition which can be administered directly, in particular parenterally. The present invention therefore also relates to an aqueous pharmaceutical composition of N-diaminomethylene-2-methyl-4,5-di(methylsulfonyl)benzamide obtainable by reconstitution of the lyophilisate according to the invention with an aqueous solvent.

The reconstituted aqueous pharmaceutical composition preferably has a pH of 1 to 3.8, preferably a pH of 2.5 to 3.5, particularly preferably a pH of about 3.

The examples explain the invention without being restricted thereto.

If concentrations are indicated in % for solutions above and below, this in each case means w/v.

### EXAMPLE 1

### Lyophilisate from Aqueous Solution Comprising

100 mg of N-diaminomethylene-2-methyl-4,5-di(methylsulfonyl)benzamide

0.11 mmol/l of mannitol

to 10 ml of water for injection purposes

adjusted to pH 3 using 1N hydrochloric acid. The reconstitution can be carried out in 5 to 100 ml of reconstitution medium, particularly preferably in 10 to 20 ml, with, for example, water for injection purposes, 0.9% sodium chloride solution or 5% glucose solution.

Preparation of the Aqueous Solution

The ingredients are dissolved in water for injection purposes in any desired sequence. The resultant solution is adjusted to the desired pH using 1N hydrochloric acid, sterile-filtered, transferred into vials under low-germ conditions and provided with stoppers.

Freeze Drying

The vials provided with stoppers are placed in the lyophilisator and frozen at −50° C. for 3 hours. The frozen solutions are subsequently subjected to a lyophilisation process in accordance with the following table:

| Time (hours) | Temperature (° C.) | Pressure (μbar) |
|---|---|---|
| 25 | −50 | 100 |
| 15 | +25 | 1 |

After completion of the freeze drying, the vacuum is removed with simultaneous introduction of nitrogen, the vials are sealed by lowering the stoppers, removed from the lyophilisator after opening the latter under low-germ conditions, and crimped.

### EXAMPLE 2 (COMPARATIVE EXAMPLE)

### Lyophilisate from Aqueous Solution Comprising

100 mg of N-diaminomethylene-2-methyl-4,5-di(methylsulfonyl)benzamide

0.055 mmol/l of mannitol

to 20 ml of water for injection purposes

adjusted to pH 4 using 1N hydrochloric acid

The preparation and reconstitution of the lyophilisate is carried out analogously to Example 1.

### EXAMPLE 3

### Lyophilisate from Aqueous Solution Comprising

100 mg of N-diaminomethylene-2-methyl-4,5-di(methylsulfonyl)benzamide

0.027 mmol/l of glucose

to 20 ml of water for injection purposes

Without adjustment of the pH (pH=3.6)

The preparation and reconstitution of the lyophilisate is carried out analogously to Example 1.

### EXAMPLE 4

### Lyophilisate from Aqueous Solution Comprising

100 mg of N-diaminomethylene-2-methyl-4,5-di(methylsulfonyl)benzamide

0.058 mmol/l of sucrose

to 20 ml of water for injection purposes

Without adjustment of the pH (pH=3.8)

The preparation and reconstitution of the lyophilisate is carried out analogously to Example 1.

### EXAMPLE 5

### Lyophilisate from Aqueous Solution Comprising

100 mg of N-diaminomethylene-2-methyl-4,5-di(methylsulfonyl)benzamide

0.056 mmol/l of lactose

to 20 ml of water for injection purposes

adjusted to pH 3.7 using 1N hydrochloric acid

The preparation of the lyophilisate is carried out analogously to Example 1.

Investigations of the Stability of the Compositions

The stability of the compositions according to the invention is tested in stability studies. To this end, the lyophilisates prepared are stored at various temperatures, removed from storage at certain times and investigated using suitable analytical methods. The climatic conditions selected are 25° C. with a relative atmospheric humidity (RH) of 60% and 40° C. with an RH of 75%. Whereas the former condition stands for storage at room temperature, the latter condition is selected as stress condition in order rapidly to achieve differences with respect to stability for the various formulations. Possible instabilities are evident in the case of N-diaminomethylene-2-methyl-4,5-di(methylsulfonyl)benzamide principally in the formation of degradation products.

Analytical Test Methods:

The lyophilisates prepared are assessed visually for the appearance of the lyophilisate cakes. The reconstitution time is likewise investigated. The formulations prepared are investigated visually with the aid of a cold-light source for particles and for the occurrence of possible turbidity.

Identity, purity and assay of the formulations comprising N-diaminomethylene-2-methyl-4,5-di(methylsulfonyl)benzamide are determined by HPLC chromatography with UV detection in a high-gradient system using eluent mixtures comprising a buffer solution and acetonitrile.

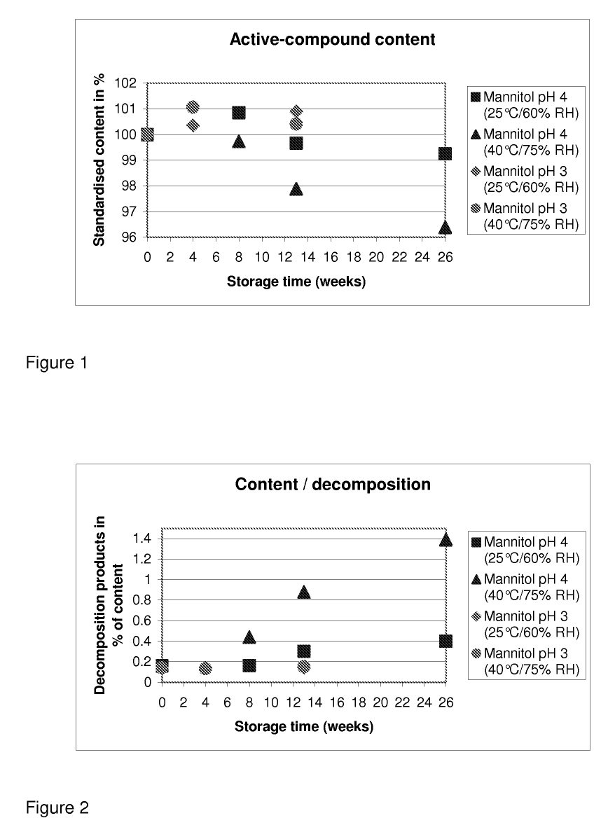

The lyophilisates obtained in accordance with Examples 1 and 2 are reconstituted by addition of 5 to 100 ml, but particularly preferably 10 to 20 ml of water for injection purposes or 5% glucose solution and investigated for their contents of N-diaminomethylene-2-methyl-4,5-di(methylsulfonyl)-benzamide and the decomposition products formed. The results obtained are shown in FIGS. 1 to 3.

FIG. 1 shows the change in the proportions, in each case standardised to 100%, of N-diaminomethylene-2-methyl-4,5-di(methylsulfonyl)benzamide in the reconstituted solutions as a function of the storage duration under the various climatic conditions (25° C./60% RH and 40° C./75% RH).

FIG. 2 shows the increase in the hydrolytically formed decomposition product 2-methyl-4,5-dimethylsulfonylbenzoic acid in the reconstituted solutions as a function of the storage duration under the various climatic conditions (25° C./60% RH and 40° C./75% RH).

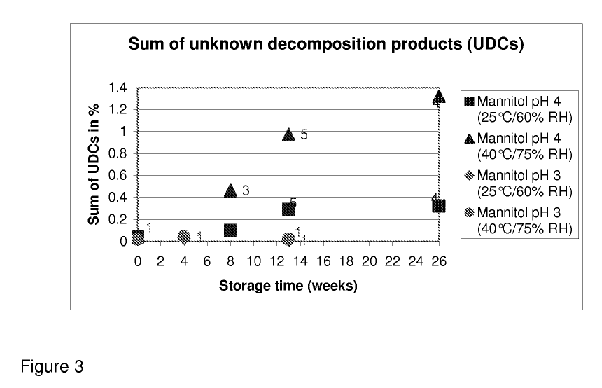

FIG. 3 shows the increase in the sum of unknown decomposition products in the reconstituted solutions as a function of the storage duration under the various climatic conditions (25° C./60% RH and 40° C/75% RH). The number of unknown decomposition products based on the measurement points is in each case indicated as a figure.

As is evident from the figures, the solution reconstituted from the lyophilisate in accordance with Example 1 has significantly higher chemical stability than the solution reconstituted from the lyophilisate. The teaching according to the invention thus leads not only to surprisingly higher physical stability, but also to surprisingly higher chemical stability of the lyophilisate.

1. Lyophilisate, obtainable by freeze drying an aqueous solution which comprises at least N-diaminomethylene-2-methyl-4,5-di(methylsulfonyl)benzamide and a bulking agent and has a pH of 1 to 3.8.

2. Lyophilisate according to claim 1, characterised in that the aqueous solution used for the freeze drying has a pH of 2.5 to 3.5.

3. Lyophilisate according to claim 1, characterised in that the aqueous solution used for the freeze drying has a pH of about 3.

4. Lyophilisate according to claim 1, characterised in that the pH of the aqueous solution used for the freeze drying is adjusted using a physiologically tolerated organic or inorganic acid.

5. Lyophilisate according to claim 4, characterised in that hydrochloric acid is employed for adjusting the pH.

6. Lyophilisate according to claim 1, characterised in that the bulking agent(s) present is/are a sugar and/or a sugar alcohol.

7. Lyophilisate according to claim 6, characterised in that the bulking agent(s) present is/are sucrose, lactose, maltose, trehalose, mannitol and/or sorbitol, preferably mannitol and/or lactose.

8. Lyophilisate according to claim 1, characterised in that one or more buffer(s) is/are furthermore present.

9. Lyophilisate according to claim 8, characterised in that the buffer(s) present is/are citrate and/or phosphate buffer(s).

10. Lyophilisate according to claim 1, characterised in that one or more physiologically tolerated adjuvants selected from the group consisting of antioxidants, preservatives and/or solubilisers are present.

11. Process for the preparation of a lyophilised pharmaceutical composition according to claim 1, characterised in that an aqueous composition at least comprising N-diaminomethylene-2-methyl-4,5-di(methylsulfonyl)benzamide and a bulking agent is prepared, the solution is if necessary adjusted to a pH of 1 to 3.8 using an acid and subsequently lyophilised.

12. Aqueous pharmaceutical composition of N-diaminomethylene-2-methyl-4,5-di(methylsulfonyl)benzamide obtainable by reconstitution of the lyophilisate according to claim 1 with an aqueous solvent.
